(19) United States
(12) Patent Application Publication
(10) Pub. No.: US 2015/0174250 A1
(43) Pub. Date: Jun. 25, 2015

(54) Composition And Method

(71) Applicants: Lee Griffiths, Hull (GB); Malcolm Tom McKechnie, Hull (GB); Steven Armes, Sheffield (GB); Adam Blanazs, Sheffield (GB)

(72) Inventors: Lee Griffiths, Hull (GB); Malcolm Tom McKechnie, Hull (GB); Steven Armes, Sheffield (GB); Adam Blanazs, Sheffield (GB)

(73) Assignee: RECKITT & COLMAN (OVERSEAS) LIMITED, Berkshire (GB)

(21) Appl. No.: 14/129,098

(22) PCT Filed: Jun. 29, 2012
(86) PCT No.: PCT/GB2012/051523

§ 371 Date: Sep. 16, 2014

(30) Foreign Application Priority Data
Jul. 1, 2011 (GB) 1111216.6

(51) Int. Cl. A61K 47/32; C11D 3/37; C11D 17/00

(52) U.S. Cl. CPC A61K 47/32; C11D 17/0017; C11D 3/3765; C11D 3/3773

(57) ABSTRACT

The present invention is directed to a method of delivering an active agent to a locus using a polymersome containing composition. The composition comprises a plurality of polymersome vesicles and a liquid matrix. The polymersome vesicles are formed in a process which comprises a cross-linking step.

The present invention relates to a method of delivering an active agent to a locus and to a method of preparing a composition for use in same.

According to a first aspect of the invention there is provided a method of delivering an active agent to a locus using a polymersome containing composition, wherein the composition comprises a plurality of polymersome vesicles and a liquid matrix, characterised in that the polymersome vesicles are formed in a process which comprises a cross-linking step.

Surprisingly it has been found that the cross-linked polymersomes display a high level of stability in the presence of a surfactant. This makes the polymersomes particularly useful in environments where surfactants are present and more particularly in environments where surfactants are purposely added, e.g. within a cleaning/detergent formulation and/or as part of a cleaning process. Typically the composition can further comprise a surfactant.

According to a second aspect of the invention there is provided a composition for use in delivering an active agent to a locus comprising plurality of polymersome vesicles and a liquid matrix, characterised in that the polymersome vesicles are formed in a process which comprises a cross-linking step.

According to a third aspect of the invention there is provided a method of delivering an active agent to a locus using a polymersome containing composition, wherein the composition comprises a plurality of polymersome vesicles, a surfactant and a liquid matrix, characterised in that the polymersome vesicles are formed in a process which comprises a cross-linking step.

According to a fourth aspect of the present invention there is provided a composition for use in delivering an active agent to a locus comprising a plurality of polymersome vesicles, a surfactant and a liquid matrix, characterised in that the polymersome vesicles are formed in a process which comprises a cross-linking step.

Preferably the active agent is pseudoepherine, ibuprofen (and its salt forms), flurbiprofen (and its salt forms), ketoprofen (and its salt forms), diclofenac (and its salt forms), paracetemol, an enzymes, a bleach, a bleach activators, a polymer.

Preferably the polymersome vesicles are capable of being disrupted.

Preferably the disruption mechanism is a chemical and/or mechanical disruption. Preferred disruption mechanisms include the application of mechanical shear and/or change in osmotic potential.

Preferably the method is for use in treating a condition.

“Polymersomes” are vesicles, which are assembled from synthetic multi-block polymers in aqueous solutions. Unlike liposomes, a polymersome does not include lipids or phospholipids as its majority component. Consequently, polymersomes can be thermally, mechanically, and chemically distinct and, in particular, more durable and resilient than the most stable of lipid vesicles. The polymersomes assemble during processes of lamellar swelling, e.g., by film or bulk rehydration or through an additional phoresis step, as described below, or by other known methods. Like liposomes, polymersomes form by “self assembly,” a spontaneous, entropy-driven process of preparing a closed semi-permeable membrane.

Because of the perselectivity of the bilayer, materials may be “encapsulated” in the aqueous interior (lumen) or intercalated into the hydrophobic membrane core of the polymersome vesicle, forming a “loaded polymersome.” Numerous technologies can be developed from such vesicles, owing to the numerous unique features of the bilayer membrane and the broad availability of super-amphiphiles, such as diblock, triblock, or other multi-block copolymers.

The synthetic polymersome membrane can exchange material with the “bulk,” i.e., the solution surrounding the vesicles. Each component in the bulk has a partition coefficient, meaning it has a certain probability of staying in the bulk, as well as a probability of remaining in the membrane. Conditions can be predetermined so that the partition coefficient of a selected type of molecule will be much higher within a vesicle's membrane, thereby permitting the polymersome to decrease the concentration of a molecule, such as cholesterol, in the bulk. In a preferred embodiment, phospholipid molecules have been shown to incorporate within polymersome membranes by the simple addition of the phospholipid molecules to the bulk. In the alternative, polymersomes can be formed with a selected molecule, such as a hormone, incorporated within the membrane, so that by controlling the partition coefficient, the molecule will be released into the bulk when the polymersome arrives at a destination having a higher partition coefficient.

Polymersomes may be formed from synthetic, amphiphilic copolymers. An “amphiphilic” substance is one containing both polar (water-soluble) and hydrophobic (water-insoluble) groups. “Polymers” are macromolecules comprising connected monomeric units. The monomeric units may be of a single type (homogeneous), or a variety of types (heterogeneous). The physical behavior of the polymer is dictated by several features, including the total molecular weight, the composition of the polymer (e.g., the relative concentrations of different monomers), the chemical identity of each monomeric unit and its interaction with a solvent, and the architecture of the polymer (whether it is single chain or branched chains). For example, in polyethylene glycol (PEG), which is a polymer of ethylene oxide (EO), the chain lengths which, when covalently attached to a phospholipid, optimize the circulation life of a liposome, is known to be in the approximate range of 34-114 covalently linked monomers (EO34 to EO114).

The preferred class of polymer selected to prepare the polymersomes is the “block copolymer.” Block copolymers are polymers having at least two, tandem, interconnected regions of differing chemistry. Each region comprises a repeating sequence of monomers. Thus, a “diblock copolymer” comprises two such connected regions (A-B); a “triblock copolymer,” three (A-B-C), etc. Each region may have its own chemical identity and preferences for solvent. Thus, an enormous spectrum of block chemistries is theoretically possible, limited only by the acumen of the synthetic chemist.

In the “melt” (pure polymer), a diblock copolymer may form complex structures as dictated by the interaction between the chemical identities in each segment and the molecular weight. The interaction between chemical groups in each block is given by the mixing parameter or Flory interaction parameter, [chi], which provides a measure of the energetic cost of placing a monomer of A next to a monomer of B. Generally, the segregation of polymers into different ordered structures in the melt is controlled by the magnitude of [chi]N, where N is proportional to molecular weight. For example, the tendency to form lamellar phases with block copolymers in the melt increases as [chi]N increases above a threshold value of approximately 10.

A linear diblock copolymer of the form A-B can form a variety of different structures. In either pure solution (the melt) or diluted into a solvent, the relative preferences of the A and B blocks for each other, as well as the solvent (if present) will dictate the ordering of the polymer material. In the melt, numerous structural phases have been seen for simple AB diblock copolymers.

To form a stable membrane in water, the absolute minimum requisite molecular weight for an amphiphile must exceed that of methanol HOCH₃, which is undoubtedly the smallest canonical amphiphile, with one end polar (HO—) and the other end hydrophobic (—CH₃). Formation of a stable lamellar phase more precisely requires an amphiphile with a hydrophilic group whose projected area, when viewed along the membrane's normal, is approximately equal to the volume divided by the maximum dimension of the hydrophobic portion of the amphiphile (Israelachvili, in Intermolecular and Surface Forces, 2 less than nd ed., Pt3 (Academic Press, New York) 1995).

The most common lamellae-forming amphiphiles also have a hydrophilic volume fraction between 20 and 50 percent. Such molecules form, in aqueous solutions, bilayer membranes with hydrophobic cores never more than a few nanometers in thickness. The present invention relates to polymserosmes with all super-amphiphilic molecules which have hydrophilic block fractions within the range of 20-50 percent by volume and which can achieve a capsular state. The ability of amphiphilic and super-amphiphilic molecules to self-assemble can be largely assessed, without undue experimentation, by suspending the synthetic super-amphiphile in aqueous solution and looking for lamellar and vesicular structures as judged by simple observation under any basic optical microscope or through the scattering of light.

For typical phospholipids with two acyl chains, temperature can affect the stability of the thin lamellar structures, in part, by determining the volume of the hydrophobic portion. In addition, the strength of the hydrophobic interaction, which drives self-assembly and is required to maintain membrane stability, is generally recognized as rapidly decreasing for temperatures above approximately 50° C. Such vesicles generally are not able to retain their contents for any significant length of time under conditions of boiling water.

Upper limits on the molecular weight of synthetic amphiphiles which form single component, encapsulating membranes clearly exceed the many kilodalton range, as concluded from the work of Discher et al., (1999).

Block copolymers with molecular weights ranging from about 2 to 10 kilograms per mole can be synthesized and made into vesicles when the hydrophobic volume fraction is between about 20 percent and 50 percent. Diblocks containing polybutadiene are prepared, for example, from the polymerization of butadiene in cyclohexane at 40[deg.] C. using sec-butyllithium as the initiator. Microstructure can be adjusted through the use of various polar modifiers. For example, pure cyclohexane yields 93 percent 1.4 and 7 percent 1.2 addition, while the addition of THF (50 parts per Li) leads to 90 percent 1.2 repeat units. The reaction may be terminated with, for example, ethyleneoxide, which does not propagate with a lithium counterion and HCl, leading to a monofunctional alcohol. This PB—OH intermediate, when hydrogenated over a palladium (Pd) support catalyst, produces PEE-OH. Reduction of this species with potassium naphthalide, followed by the subsequent addition of a measured quantity of ethylene oxide, results in the PEO-PEE diblock copolymer. Many variations on this method, as well as alternative methods of synthesis of diblock copolymers are known in the art; however, this particular preferred method is provided by example, and one of ordinary skill in the art would be able to prepare any selected diblock copolymer.

For example, if PB-PEO diblock copolymers were selected, the synthesis of PB-PEO differs from the previous scheme by a single step, as would be understood by the practitioner. The step by which PB—OH is hydrogenated over palladium to form PEO-OH is omitted. Instead, the PB—OH intermediate is prepared, then it is reduced, for example, using potassium naphthalide, and converted to PB-PEO by the subsequent addition of ethylene oxide.

In yet another example, triblock copolymers having a PEO end group can also form polymersomes using similar techniques. Various combinations are possible comprising, e.g., polyethylene, polyethylethylene, polystyrene, polybutadiene, and the like. For example, a polystyrene (PS)-PB-PEO polymer can be prepared by the sequential addition of styrene and butadiene in cyclohexane with hydroxyl functionalization, re-initiation and polymerization. PB-PEE-PEO results from the two-step polymerization of butadiene, first in cyclohexane, then in the presence of THF, hydrolyl functionalization, selective catalytic hydrogenation of the 1.2 PB units, and the addition of the PEO block.

A plethora of molecular variables can be altered with these illustrative polymers, hence a wide variety of material properties are available for the preparation of the polymersomes. ABC triblocks can range from molecular weights of 3,000 to at least 30,000 g/mol. Hydrophilic compositions should range from 20-50 percent in volume fraction, which will favor vesicle formation.

The molecular weights must be high enough to ensure hydrophobic block segregation to the membrane core. The Flory interaction parameter between water and the chosen hydrophobic block should be high enough to ensure said segregation. Symmetry can range from symmetric ABC triblock copolymers (where A and C are of the same molecular weight) to highly asymmetric triblock copolymers (where, for example, the C block is small, and the A and B blocks are of equal length).

The polymersomes are preferably based on A PBd-PEO copolymer. Alternative polymers include poly(hexyl methacrylate)-block-poly[2-(dimethylamino)ethyl methacrylate] (PHMA-PDMA), poly(hexyl methacrylate)-block-poly(methacrylic acid) (PHMA-PMAA), poly(butyl methacrylate)-block-poly(methacrylic acid) (PBMA-PMAA), poly(ethylene oxide)-block-poly(hexyl methacrylate) (PEO-PHMA), poly(butyl methacrylate)-block-poly[2-(dimethylamino)ethyl methacrylate (PBMA-PDMA), poly(hexyl methacrylate)-block-poly[2-(dimethylamino)ethyl methacrylate (PHMA-PDMA), poly(butyl methacrylate)-block-Poly(ethylene oxide) (PBMA-PEO).

A surfactant or surface active agent is well-known and is a substance which lowers the surface tension of the medium in which it is dissolved, and/or the interfacial tension with other phases, and, accordingly, is positively adsorbed at the liquid/vapour and/or at other interfaces. The term surfactant is also applied correctly to sparingly soluble substances, which lower the surface tension of a liquid by spreading spontaneously over its surface. Some of the more common and typical surfactants are as listed below (not inclusive)

A soap is a salt of a fatty acid, saturated or unsaturated, containing at least eight carbon atoms or a mixture of such salts.

A detergent is a surfactant (or a mixture containing one or more surfactants) having cleaning properties in dilute solution (soaps are surfactants and detergents).

A syndet is a synthetic detergent; a detergent other than soap. An emulsifier is a surfactant which when present in small amounts facilitates the formation of an emulsion, or enhances its colloidal stability by decreasing either or both of the rates of aggregation and coalescence.

A foaming agent is a surfactant which when present in small amounts facilitates the formation of a foam, or enhances its colloidal stability by inhibiting the coalescence of bubbles.

The property of surface activity is usually due to the fact that the molecules of the substance are amphipathic or amphiphilic, meaning that each contains both a hydrophilic and a hydrophobic (lipophilic) group.

These surface active species are commonly used for example in cleaning formulations, as emulsifiers, solubilisers, hydrotropes, foaming agents, wetting agents, dispersants, as structured systems for modifying rheology, however any molecule with the above properties can be a surfactant. This may include some of the pharmaceutical actives of relevance within this filing e.g the propionic acid derivative class of NSAID's such as flurbiprofen and ibuprofen.

Generally (following synthesis) such a polymer is used to form polymersomes (vesicles).

The present invention will now be described in more detail with reference to the accompanying Figures in which:

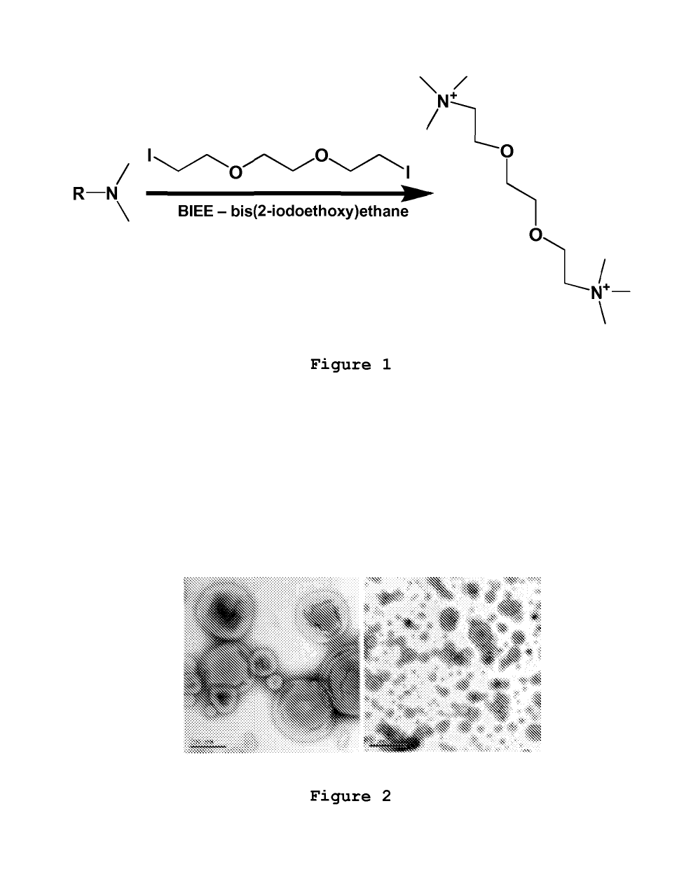

FIG. 1 illustrates the covalent cross-linking of the PHMA-PDMA polymersomes is conducted through the reaction of bi-functional bis(2-idoethoxy)ethane within the DMA residues;

FIG. 2 illustrates TEM micrographs of BIEE cross-linked PHMA62-PDMA30 polymersomes;

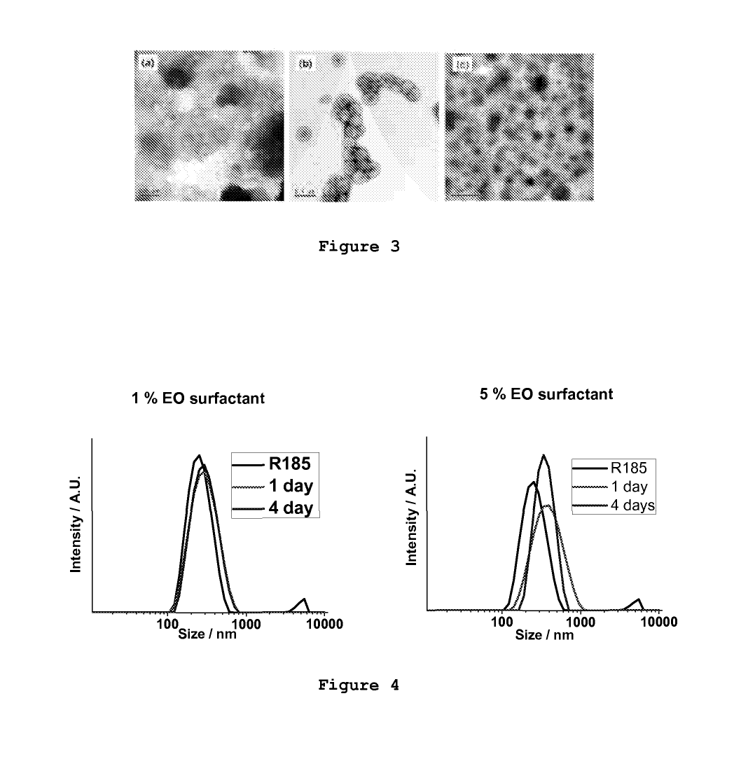

FIG. 3 illustrates BIEE cross-linked PHMA62-PDMA30 polymersomes 5 hours after surfactant treatment at 5 w/v % using (a) cationic (didecylmethylamomiun chloride—DDMAC) (b) anionic (dodecylbenzene sulfonic acid 88% sodium salt) and (c) charge neutral (alcohol ethoxylate: 7EO C12-C14); and

FIG. 4 illustrates DLS studies on PGMA69-PHPMA340 (0.5 w/w %) polymersomes after treatment with the non-ionic, alcohol ethoxylate surfactant (5 w/v %).

### EXAMPLES

### Surfactant Stability of PHMA-PDMA Polymersomes

The stability of 0.5 w/v % polymersome solutions generated from the solvent switch of both PHMA₆₅-PDMA₃₀ and PHMA₂₂-PDMA₁₁ were assessed with added cationic (didecylmethylamomiun chloride—DDMAC), anionic (dodecylbenzene sulfonic acid 88% sodium salt) and charge neutral (alcohol ethoxylate: 7EO C12-C14) surfactants at varying concentrations.

The effect of added surfactants to these polymersome solutions are summarised in Tables 1 and 2. The effect is measured as a loss in turbidity, as this relates to a loss in the polymersome structure. As surfactant is added to the low molecular weight polymersomes generated from PHMA₂₂-PDMA₁₁, after as little as one minute the solutions become clear (Table 1). This is due to the complete loss in polymersome structure as the surfactants solubilise the block copolymers. The higher molecular weight PHMA₆₅-PDMA₃₀ polymersomes have slightly higher resistance (Table 2). On addition of 1 w/v % surfactant all remain immediately stable, but over the course of 30 minutes a slight loss in turbidity is observed. However, after 24 h each of the solutions is almost clear. A higher surfactant concentration (5 w/v %) results in increased polymersome dissolution. After 1 minute, there is some stability as observed by a minimal loss in turbidity. However, after 30 minutes (depending on the surfactant type) a large loss in turbidity occurs. After 24 h, complete polymersome destruction is observed with the generation of clear solutions.

TABLE 1
The effect of added surfactant to 0.5 w/v % polymersome
solutions generated via a solvent switch of PHMA₂₂-
PDMA₁₁ from THF to water at pH 7.

| | | After addition | | |
|---|---|---|---|---|
| Surfactant | Conc./w/v % | 1 min | 30 min | 24 h |
| Cationic | 5 | Clear solution | — | — |
| Anionic | 5 | Clear solution | — | — |
| Neutral | 5 | Clear solution | — | — |
| Cationic | 1 | Clear solution | — | — |
| Anionic | 1 | Clear solution | — | — |
| Neutral | 1 | Clear solution | — | — |

TABLE 2
The effect of added surfactant to 0.5 w/v % polymersome
solutions generated via a solvent switch of PHMA₆₅-
PDMA₃₀ from THF to water at pH 7.

| | Conc./ | After addition | | |
|---|---|---|---|---|
| Surfactant | w/v % | 1 min | 30 min | 24 h |
| Cationic | 5 | No | Losing turbidity | Clear solution |
| | | change | | |
| Anionic | 5 | No | Slight loss | Clear solution |
| | | change | | |
| Neutral | 5 | Slight | Almost clear | Clear solution |
| | | loss | | |
| Cationic | 1 | No | Slight loss | Massive turbidity |
| | | change | | loss |
| Anionic | 1 | No | Slight loss | Clear |
| | | change | | |
| Neutral | 1 | No | Slight loss | Massive turbidity |
| | | change | | loss |

In order to increase the surfactant resistance of the PHMA-PDMA polymersomes, the DMA residues in the polymersome coronas were cross-linked using bis(2-idoethoxy)ethane (BIEE) (FIG. 1).

Table 3 shows the enhanced stability of the shell cross-linked polymersomes towards surfactant resistance. Now both the low and higher molecular weight diblock copolymersome solutions are stable over 24 h when treated with a 5 w/v % surfactant (by eye turbidity).

TABLE 3
The effect of added surfactant to 0.5 w/v % PHMA-
PDMA polymersome solutions cross-linked using BIEE.

| | Conc./ | After addition | | |
|---|---|---|---|---|
| Surfactant | w/v % | 1 min | 30 min | 24 h |
| Cationic | 5 | No Change | No | No change, but some |
| | | | change | phase sep |
| Anionic | 5 | No | No | — |
| | | Change* | change* | |
| Neutral | 5 | No | No | No change, but some |
| | | Change | change | phase sep |
| Cationic | 5 | No | No | No change, but some |
| | | Change | change | phase sep |
| Anionic | 5 | No | No | — |
| | | Change* | change* | |
| Neutral | 5 | No | No | No change, but some |
| | | Change | change | phase sep |

*Also, charge complexation occurs, with polymer precipitate present after addition of anionic surfactant

TEM micrographs in FIG. 2 show polymersome structure is maintained after cross-linking.

To assess whether polymersome structure was retained after surfactant treatment, TEM studies were performed of the cross-linked polymersomes with 5 w/v % of either cationic (didecylmethylamomiun chloride—DDMAC), anionic (dodecylbenzene sulfonic acid 88% sodium salt) or charge neutral (alcohol ethoxylate: EO7-(C12-C14)).

As observed in FIG. 3, polymersome morphology appears to be retained for the polymersomes treated with each surfactant after 5 hrs. As a note, the polymersomes dissolute almost instantaneously without cross-linking, leaving a clear solution.

PGMA-PHPMA Polymersome Stability from the In-Situ Generation Method

PGMA-PHPMA polymersomes were also tested for stability from each of the three surfactants. Each surfactant was mixed at both 1 and 5 w/w % in polymersome solutions formed from PGMA69-PHPMA340 (0.5 w/v %). Immediately after anionic or cationic surfactant addition polymersome dissolution occurred. However, the same experiment using the non-ionic alcohol ethoxylate surfactant yields no significant change in the size measured by DLS over several days (FIG. 4). This suggests the PGMAPHPMA polymersomes are stable to this surfactant, possibly due to repulsive interactions between the hydroxyl groups in the PGMA corona and the ethoxylated surfactant groups. No significant deviation in size or scattered light intensity is observed, indicating good stability to the surfactant challenge.

1. A method of delivering an active agent to a locus comprising:
delivering an active agent to a locus using a polymersome containing composition;
wherein the composition comprises a plurality of polymersome vesicles and a liquid matrix; and
wherein the polymersome vesicles are formed in a process which comprises a cross-linking step.

2. The method according to claim 1, wherein the polymersome vesicles are capable of being disrupted by the application of mechanical shear.

3. The method according to claim 1, wherein the polymersome vesicles are capable of being disrupted by one or more chemical and osmotic potential disruption.

4. The method according to claim 1, wherein the method is for use in treating a substrate/surface.

5. The method according to claim 1, wherein the polymersome is a vesicle formed from an amphilic di-block copolymer.

6. The method according to claim 1, wherein the cross-linking step comprises a cross-linking agent comprising glycidyl methacrylate, the cross-linking agent present in an amount of between 0.5 and 2.0 Mole equivalents with respect to the epoxy group in the glycidyl methacrylate.

7. The method according to claim 1, wherein the cross-linking step comprises a cross-linking agent comprising one or more of bis(2-idoethoxy)ethane (BIEE), and an amine base.

8. The method according to claim 1, wherein the concentration of polymersome is 0.5-1% by weight of the composition.

9. The method according to claim 2, wherein the amount of shear is from 0.5-50 Pa.

10. The method according to claim 2, wherein the time needed for shear is less than 10 minutes.

11. A composition for use in delivering an active agent to a locus comprising:
a plurality of polymersome vesicles; and
a liquid matrix;
wherein the polymersome vesicles are formed in a process which comprises a cross-linking step.

12. The composition of claim 11 further comprising a surfactant.

13. The method claim 1, wherein the composition further comprises a surfactant.

14-16. (canceled)

17. The method according to claim 1, wherein the polymersome is a vesicle formed from an admixture of polybutadiene (PBd) and polyethylene oxide (PEO) copolymers.

18. (canceled)

19. The method according to claim 1, wherein the cross-linking agent comprises an amine base selected from the group consisting of Ethylene Diamine and Jeffamine.

20. (canceled)

21. The method according to claim 2, wherein the amount of shear is from 0.5-10 Pa.

22. The method according to claim 2, wherein the time needed for shear is less than 3 minutes.

23. (canceled)

24. A method of delivering an active agent to a locus comprising:
forming polymersome vesicles in a process comprising a cross-linking step; and
delivering an active agent to a locus using a polymersome containing composition comprising a plurality of the formed polymersome vesicles and a liquid matrix.

25. The method according to claim 24, wherein the polymersome containing composition further comprises a surfactant, and the polymersome vesicles are capable of being disrupted by the application of mechanical shear.

26. The method according to claim 25, wherein the cross-linking step comprises a cross-linking agent comprising glycidyl methacrylate, the cross-linking agent present in an amount of between 0.5 and 2.0 Mole equivalents with respect to the epoxy group in the glycidyl methacrylate.

27. The method according to claim 25, wherein the cross-linking step comprises a cross-linking agent comprising one or more of bis(2-idoethoxy)ethane (BIEE) and an amine base.

28. The method according to claim 25, wherein the concentration of polymersome is 0.5-1% by weight of the composition.

29. The method according to claim 25, wherein the amount of shear is from 0.5-10 Pa; and
wherein the time needed for shear is less than 3 minutes.
